(19) United States
(12) Patent Application Publication
(10) Pub. No.: US 2020/0108270 A1
(43) Pub. Date: Apr. 9, 2020

(54) STROBOSCOPIC LIGHT THERAPY FOR TREATMENT OF MEMORY LOSS RELATED TO ALZHEIMER'S DISEASE, METHOD AND SYSTEM

(71) Applicant: Heugenis Inc., Grand Rapids, MI (US)

(72) Inventors: Kim LaVine, Muskegon, MI (US); Chokha Palayamkottai, Grand Rapids, MI (US)

(21) Appl. No.: 16/593,822

(22) Filed: Oct. 4, 2019

Related U.S. Application Data
(60) Provisional application No. 62/742,171, filed on Oct. 5, 2018.

(51) Int. Cl. A61N 5/06; G02C 7/10; G02F 1/137; G02C 11/00; G06F 3/16

(52) U.S. Cl. CPC A61N 5/0618; G02C 7/101; G02F 1/137; G02C 11/10; G06F 3/16; H04W 76/10; A61N 2005/0629; A61N 2005/0632; A61N 2005/0648; A61N 2005/0657; A61N 5/0622

(57) ABSTRACT

A wireless, programmable stroboscopic eyewear device is used to clear amyloid proteins from the brain. The device employs passive, non-invasive stroboscopic lens technology to combat buildup of amyloid proteins in the brain by improving gamma oscillations in the brain. The wearable device delivers non-invasive stroboscopic stimulation in the 20-60 Hz range. Treatment sessions are for an average of twenty minutes a day, seven days a week through passive use. The wearable device transmits data to a smartphone based mobile application, as a means to encourage an effective treatment protocol as determined and monitored by the user's physicians and caregivers. The stroboscopic effect is created through the synchronized opening and closing of Liquid Crystal Display (LCD) lenses which electronically switch from clear to opaque, utilizing noninvasive ambient light to create the strobe effect, as opposed to invasive flashing lights.

### CROSS-REFERENCE TO RELATED APPLICATIONS

This application claims benefit of U.S. provisional patent application Ser. No. 62/742,171, filed Oct. 5, 2018, the entirety of which is incorporated herein by this reference thereto.

### BACKGROUND

### Technical Field

The present disclosure is related to treatment for cognitive impairment. More particularly, the present disclosure is directed to methods and system using stroboscopic light therapy for treatment of memory loss related to Alzheimer's disease.

### Background Information

### About Alzheimer's Disease

The healthy human brain contains tens of billions of neurons—specialized cells that process and transmit information via electrical and chemical signals. They send messages between different parts of the brain, and from the brain to the muscles and organs of the body. Alzheimer's disease disrupts this communication among neurons, resulting in loss of function and cell death (http://www.nia.nih.gov/health/what-happens-brain-alzheimers-disease).

Unlike many cells in the body, which are relatively short-lived, neurons have evolved to live a long time—more than 100 years in humans. As a result, neurons must constantly maintain and repair themselves. Neurons also continuously adjust, or “remodel,” their synaptic connections depending on how much stimulation they receive from other neurons. For example, they may strengthen or weaken synaptic connections, or even break down connections with one group of neurons and build new connections with a different group. Adult brains may even generate new neurons—a process called neurogenesis. Remodeling of synaptic connections and neurogenesis are important for learning, memory, and possibly brain repair.

Many molecular and cellular changes take place in the brain of a person with Alzheimer's disease. These changes can be observed in brain tissue under the microscope after death. The brain typically shrinks to some degree in healthy aging but, surprisingly, does not lose neurons in large numbers. In Alzheimer's disease, however, damage is widespread, as many neurons stop functioning, lose connections with other neurons, and die. Alzheimer's disrupts processes vital to neurons and their networks, including communication, metabolism, and repair.

Amyloid Plaques: The beta-amyloid protein involved in Alzheimer's comes in several different molecular forms that collect between neurons. It is formed from the breakdown of a larger protein, called amyloid precursor protein. One form, beta-amyloid 42, is thought to be especially toxic. In the Alzheimer's brain, abnormal levels of this naturally occurring protein clump together to form plaques that collect between neurons and disrupt cell function.

### About Stroboscopic Technology

Clinical and university studies have resulted in the adoption of stroboscopic technology for improving cognitive function in a range of subject, including Mir astronauts and current-day superstar athletes.

NASA research has revealed that stroboscopic goggles that simulate a strobe-lighting effect can prevent the nauseating effects of space sickness—and that of more down-to-Earth travel.

Researchers funded by the National Science Foundation, tested the theory that effective learning depends on the formation of perceptual-motor feedback loops, so-called “perceptual traces.” They used strobe lights flashing at 2, 4, 10, 15 and 20 times a second to distract subjects from a task requiring precise eye-hand coordination. The more sensory feedback that subjects received, the better they did. Even more intriguing, performance improved at faster strobe rates of 10, 15 and 20/seconds—but not at slower flashes at 2 and 5/second.

Professional athletes have been shown to possess an extraordinary ability to learn complex and dynamic visual scenes, far better than amateur athletes or non-athletes. Past a certain threshold of sensory distraction, trainees could fill in missing details by instinct or experience; in effect, they learned how to visualize an unfolding event by drawing on their previous knowledge.

+

### SUMMARY

A wireless, programmable stroboscopic eyewear device is used to clear amyloid proteins from the brain. The device employs passive, non-invasive stroboscopic lens technology to combat buildup of amyloid proteins in the brain by improving gamma oscillations in the brain. The wearable device delivers non-invasive stroboscopic stimulation in the 20-60 Hz range. Treatment sessions are for an average of twenty minutes a day, seven days a week through passive use. The wearable device transmits data to a smartphone based mobile application, as a means to encourage an effective treatment protocol as determined and monitored by the user's physicians and caregivers. The stroboscopic effect is created through the synchronized opening and closing of Liquid Crystal Display (LCD) lenses which electronically switch from clear to opaque, utilizing noninvasive ambient light to create the strobe effect, as opposed to invasive flashing light.

In embodiments, the device measures data from the user and integrates it into a medical monitoring portal 310 that is accessible to doctors.

In embodiments, the stroboscopic effect is created through the synchronized opening and closing of Liquid Crystal Display (LCD) lenses which electronically switch from clear to opaque, utilizing noninvasive ambient light to create the strobe effect as opposed to invasive flashing lights.

In embodiments, the wearable device is designed to transmit data to a smartphone based mobile application, as a means to encourage an effective treatment protocol as determined and monitored by the user's physicians and caregivers.

In embodiments, prophylactic use of the device before an individual shows signs of early onset memory loss is recommended, as stroboscopic research has demonstrated enhanced cognitive processing skills through “temporal-occlusion training.” It works by temporarily blocking the view of the action, so the trainee has to “guess” what's going to happen. Academic research into the benefits of this vision training revealed that stroboscopic training led to significantly greater re-test improvement in central visual field motion sensitivity and transient attention abilities.

### BRIEF DESCRIPTION OF THE DRAWINGS

FIGS. 1A and 1B provide views of of an exemplary pair of stroboscopic eyewear;

FIG. 2 provides a schematic of a data network configured to support the device of FIGS. 1A and 1B;

FIG. 3 provides a diagram of a web application interface for providing a system of patient care incorporating the eyewear of FIG. 1;

FIG. 4 provides a block diagram of a computational device upon which a controller for the device of FIG. 1 is implemented;

FIG. 5 provides a block diagram of a system for a healthcare and Alzheimer's treatment management system;

FIGS. 6 and 7 provide diagrams of a process and system for automatically maintaining a EHR (electronic health record) with device data for a patient that is collected during a medically-related event automatically; and

FIGS. 8 and 9 provide diagrams of a process and system for automatically maintaining a EHR (electronic health record) with device data for a patient that is collected during a medically-related event automatically in a hospital setting.

### DETAILED DESCRIPTION

A wireless, programmable stroboscopic eyewear device is used to clear amyloid proteins from the brain. The device employs passive, non-invasive stroboscopic lens technology to combat buildup of amyloid proteins in the brain by improving gamma oscillations in the brain. The wearable device delivers non-invasive stroboscopic stimulation in the 20-60 Hz range. Treatment sessions are for an average of twenty minutes a day, seven days a week through passive use. The wearable device transmits data to a smartphone based mobile application, as a means to encourage an effective treatment protocol as determined and monitored by the user's physicians and caregivers. The stroboscopic effect is created through the synchronized opening and closing of Liquid Crystal Display (LCD) lenses which electronically switch from clear to opaque, utilizing noninvasive ambient light to create the strobe effect, as opposed to invasive flashing lights.

In embodiments, the device measures data from the user and integrates it into a medical monitoring portal 310 that is accessible to doctors.

In embodiments, the stroboscopic effect is created through the synchronized opening and closing of Liquid Crystal Display (LCD) lenses which electronically switch from clear to opaque, utilizing noninvasive ambient light to create the strobe effect as opposed to invasive flashing lights.

In embodiments, the wearable device is designed to transmit data to a smartphone based mobile application, as a means to encourage an effective treatment protocol as determined and monitored by the user's physicians and caregivers via a patient care portal 310.

In embodiments, prophylactic use of the device before an individual shows signs of early onset memory loss is recommended as stroboscopic research has demonstrated enhanced cognitive processing skills through “temporal-occlusion training.” It works by temporarily blocking the view of the action, so the trainee has to “guess” what's going to happen. Academic research into the benefits of this vision training revealed that stroboscopic training led to significantly greater re-test improvement in central visual field motion sensitivity and transient attention abilities.

NASA

The benefits of stroboscopic technology were explored by NASA as early as 2006, when it was demonstrated that stroboscopic goggles that simulate a strobe-lighting effect could prevent the nauseating effects of space sickness—and that of more down-to-Earth travel. Designed by Millard Reschke at JSC, with George Ford and Jeffrey Somers at Wyle Laboratories in Houston, the goggles were honored at the Inventors' Luncheon 2006 at NASA's Johnson Space Center in Houston, Tex. Reschke came up with the idea for the glasses after observing a particular astronaut who had returned from a long stay on Russia's former space station, Mir. A 1981 study suggested that strobe lighting might help with motion sickness, but it was not clear why. Reschke's team noticed that the astronaut's eyes darted back and forth more than normal. The team suspected these eye jitters—known as square wave jerks—were helping to “freeze” the moving visual scene on his retina, protecting him from space sickness. After Reschke observed a Mir astronaut, he wondered whether strobe lighting might also be freezing images on the retina. So, his team created glasses with lenses made of LCD “shutters” that switch from dark to clear very quickly, providing a strobe effect. In a study published in January 2006 through the National Institute of Health, Reschke's team tested a pair of the glasses. The LCD +shutters allowed four 10-millisecond “flashes” of light to come through each second. The subjects using the glasses were able to endure simulated motion sickness for the entire 30-minute duration of the study—those without the goggles lasted only 24 minutes on average (Reschke MF1, Somers J T, Ford G. Stroboscopic Vision as a Treatment for Motion Sickness: Strobe Lighting Vs. Shutter Glasses; Neurosciences Laboratories, NASA Johnson Space Center, Houston, Tex., USA. millard.f.reschke@nasa.gov Retrieved from http://www.ncbi.nlm.nih.gov/pubmed/16422446).

National Science Foundation

More than 40 years ago, Jack A. Adams proposed that effective learning depends on the formation of perceptual-motor feedback loops, which he called “the perceptual trace” (Jack A. Adams, “A Closed-Loop Theory of Motor Learning,” Journal of Motor Behavior, 1971, 3, pages 111-149). Funded by the National Science Foundation, researchers at Saint Olaf College in Minnesota tested his theory. They used strobe lights flashing at 2, 4, 10, 15 and 20 times a second to distract subjects from a task requiring precise eye-hand coordination. Confirming Adams, the more sensory feedback that subjects received, the better they did. Even more intriguing, performance improved at faster strobe rates of 10, 15 and 20/seconds—but not at slower flashes at 2 and 5/second. And the training worked best at 15 flashes a second. The researchers wondered, was this some kind of “sweet spot?” They suggested that for a perceptual trace to form and performance to improve, the kinesthetic feedback must be backed up with reasonably precise visual information. If that visual information was not precise—because the flashes were too slow or fast—no perceptual trace could be developed (Howard Thorsheim et al, “Visual and Kinesthetic Components of Pursuit-Tracking Performance,” 1973, pages 6-7).

Drawing on research dating from 1952, scientists in 1964 advanced the theory that the+ brain has a “rhythmic scanning mechanism” running at about 100 milliseconds (M. Russell Harter, Robert Eason, Carroll White, “Effects of Intermittent Visual Input Disruption, Flicker-Rate, and Work Time on Tracking Performance and Activation Level,” Perceptual and Motor Skills, 1964, 19, page 832). This is very much like the clock in every computer, which synchronizes all the signals handled by the system to a basic frequency. Researchers reasoned they could disrupt the visual input for some interval up to 100 msec before any subjects “start losing temporal information at the neural level.” Sure enough, experiments showed that performance was significantly affected by flicker-rate. Test results got worse from 0 to 9 flashes/second—the closest interval to the brain's hypothetical sampling rate—and then improved again up to the fastest rate they studied, 24/second (Harter et al, page 840).

Many questions remained: If the brain has a “sampling rate” of 10 Hertz, how would it cope with training exercises done at faster or slower rates? Could we actually train the brain to form a perceptual trace faster? And would that improve any related visual or auditory skills? Researchers from the world of high-performance sports were already looking for those answers.

Precedents in Elite Sports Performance A recent study at the University of Montreal showed that professional athletes have an extraordinary ability to learn complex and dynamic visual scenes, far better than amateur athletes or non-athletes. The researchers concluded, “these remarkable mental processing and learning abilities should be acknowledged as critical elements for world-class performance in sport” (Jocelyn Faubert, “Professional athletes have extraordinary skills for rapidly learning complex and neutral dynamic visual scenes,” Nature Scientific Reports, January 2013, 3:1154, DOI: 10.1038/srep01154). The question is, how can elite trainers build on these strengths to counter the effects of BAR and develop true champions?

Past a certain threshold of sensory distraction, trainees fill in any missing details by instinct or experience. A recent literature survey that compared elite training methods for sports and military touched on an intriguing technique that provides a likely answer. First devised during the 1960s, this is called “temporal-occlusion training.” It works by temporarily blocking the view of the action, so the trainee has to “guess” what's going to happen. For example, in one study tennis players watched film shot from the viewpoint of a player on the court. Just as the opposing player's racket made contact with the ball, the screen would go dark. The player being trained had to decide where their opponent's shot was headed. With training, the participants significantly improved how quickly and accurately they could predict the direction of an opponent's shot (M. J. Haskins, “Development of a Response—Recognition Training Film In Tennis,” Perceptual and Motor Skills, 1965 August; 21 (1), pages 207-211).

Further studies in temporal occlusion followed, using various devices:
Film of baseball pitches and special helmets that blocked the hitter's vision as he stepped onto the plate (W. A. Burroughs, “Visual simulation training of baseball batters,” International Journal of Sports Psychology, 1984, Vol 15, pages 117-126);
More filmed tennis shots (A. M. Williams, P. Ward, J. Knowles and N.J. Smeeton, “Anticipation skill in a real-world task: Measurement, training, and transfer in tennis,” Journal; and
Near-life-size video intended to train outfielders to play goalkeeper (A. M. Williams, P. Ward, and C. Chapman, “Training perceptual skill in field hockey: Is there transfer from the laboratory to the field?” Research Quarterly for Exercise and Sport, 2003 74(1), pages+98-103).

In all cases, the athletes who practiced using temporal occlusion improved their accuracy, reaction time, or both. Even though some critical information was hidden from them during practice exercises, they still managed to acquire a perceptual trace that generated effective recall—some form of “muscle memory”—even when the occlusion was removed.

It seemed that past a certain threshold of sensory distraction, trainees could fill in missing details by instinct or experience; in effect, they learned how to visualize an unfolding event by drawing on their previous knowledge. In any event, this approach to training seemed to work. And it wasn't long before the biggest name in sports took notice.

Oregon Ducks college football team trained with prototype stroboscopic glasses for several successful seasons, then won the Rose Bowl in 2012 and the Fiesta Bowl in 2013.

Green Bay Packers receiver Greg Jennings reduced his incomplete passes from 8 per season to 3, saying the stroboscopic glasses enabled him to “see the ball a little better and react quicker” (Tim Newcomb, “Visions of Perfection,” Sports Illustrated, 9 Jan. 2012).

Florida Junior Blades hockey team trained with the prototype stroboscopic glasses and racked up an impressive record of 100 wins with only 19 losses and 5 ties, plus league records for most goals scored, fewest goals against, and most short-handed goals (“Florida Jr. Blades: Empire team celebrates reaching 100-win milestone”, USA Junior Hockey Magazine, 27 Jan. 2013).

Academic research into the benefits of this vision training continues, mainly at Duke University. Researchers there gave hundreds of participants—varsity players and nonathletes alike—both lab and field exercises wearing the prototype stroboscopic glasses, followed up by computerized visual testing. “Results revealed that stroboscopic training led to significantly greater re-test improvement in central visual field motion sensitivity and transient attention abilities,” say the researchers from Duke University (L. Gregory Appelbaum, Julia E. Schroeder, Matthew S. Cain and Stephen R. Mitroff, “Improved visual cognition through stroboscopic training,” Frontiers in Psychology, 2:276, 28 Oct. 2011). These benefits appear to be “relatively robust” since they appeared after only two days of training, affecting both varsity athletes and non-athletes alike. “Visual attention is a critical ability for many domains, and even a small increase can have profound effects,” concluded the researchers. “A small percent improvement in motion perception and focused attention may mean the world to an athlete engaged in a competitive sport” (Appelbaum et al., page 11).

Stroboscopic Research and Athletes

The dynamic aspects of sports often place heavy demands on visual processing. As such, an important goal for sports training should be to enhance visual abilities. Recent research has suggested that training in a stroboscopic environment, where visual experiences alternate between visible and obscured, may provide a means of improving attentional and visual abilities in athletes. The study explored whether stroboscopic training could impact anticipatory timing—the ability to predict where a moving stimulus will be at a specific point in time. Anticipatory timing is a critical skill for both sports and non-sports activities, and thus finding training improvements could have broad impacts. Participants completed a pre-training assessment that used a Bassin Anticipation Timer (BAT) to measure their abilities to accurately predict the timing of a moving visual stimulus. Immediately after this initial assessment, the participants completed training trials, but in one of two conditions. Those in the Control condition proceeded as before with no change. Those in the Strobe condition completed the training trials while wearing specialized eyewear that had lenses that alternated between transparent and opaque (rate of 100 ms visible to 150 ms opaque). Post-training assessments were administered immediately after training, ten minutes after training, and ten days after training. Compared to the Control group, the Strobe group was significantly more accurate immediately after training, was more likely to respond early than to respond late immediately after training and ten minutes later and was more consistent in their timing estimates immediately after training and ten minutes later.

Use of Stroboscopic Technology in Major League Baseball

In the elite world of Major League Baseball, teams who once shunned prospects who wore glasses are now using goggles to enhance player's visual training and cognitive reflexes (sports Illustrated (Apr. 13, 2015). “Seeing the Benefit: MLB Teams Focus on Enhancing Players' Visual Training.” Retrieved from http://www.si.com/edge/2015/04/21/seeing-the-benefit-mlb-vision-training-tampa-bay-rays).

Teams across baseball are working with companies like NEUROSCOUTING, LLC (Cambridge, Mass.) to introduce vision and reaction-time to their evaluation and training processes. “Hitters have three tenths of a second on a 90-mph pitch to make a decision,” says Dr. Keith Smithson, team optometrist for the Washington Nationals baseball team. “If we can buy a tenth in there somewhere, we gain the ability to foul it off if we were gonna miss it or put it in play if we were gonna foul it off.”

Smithson uses a three-tiered approach to eye care: He tests and corrects visual acuity (the average major leaguer has 20-21 [symbol] 2 vision, and he prescribes corrective lenses for anyone at or above 20-20. He also trains the seven muscles around the eye to focus through drills, both high and low tech and he tries to improve visual processing, the communication between the eyes and the brain. “It used to be that we had the science, but technology hadn't caught up,” says Smithson. “Now we're starting to have the technology too.”

That is one of the benefits of vision training—unlike with a lifting program, where an athlete might see a result only after several weeks of training, the difference can show up after 15 minutes of ocular workouts, so it's easy to get players to buy in. As often as possible, Smithson tries to incorporate vision training into other exercises, so it doesn't even take up extra time. Players wear strobe glasses that blink cloudy and clear while sprinting or they catch whiffle balls at random while doing squats.

### Use of Stroboscopic Technology in Professional Hockey

Published research proves that strobe glasses dramatically improve the performance of professional hockey players. Players who trained with special eyewear that only allowed them to see action intermittently showed significant improvement in practice drills, according to a Duke University study with the NHL's Carolina Hurricanes. Earlier research using the stroboscopic eyewear during training showed improved vision, visual attention, and ability to anticipate the timing of moving items. But the small pilot study with Hurricanes players is the first to directly explore whether those effects can improve sports performance. Players who trained with the strobe eyewear experienced an 18 percent performance improvement in a series of on-ice skill tests. A control group showed no change.

In a study conducted through Duke University, Stephen R. Mitroff collaborated with Hurricanes athletic trainers and strength and condition coaches Peter Friesen and Doug Bennett to test players during the team's 16-day pre-season training camp. Eleven players completed the full study wearing prototype stroboscopic glasses. The athletes were randomly divided into a five-player control group that completed normal training and a six-player strobe group that wore the eyewear once daily during normal training. Each group completed a performance assessment before and after training. Forwards were asked to perform a task that involved difficult skating before taking shots on goal, and defensemen were asked to skate in a circle before completing long passes. “That 18% improvement for on-ice skills for professional players is huge,” Mitroff said. “This is a dramatic improvement observed in professional athletes” (Athletic Training & Sport Health (November/December 2013). “Strobe Glasses Improve Hockey Players' Performance,” Stephen R. Mitroff, Peter Friesen, Doug Bennett, Herb Yoo, Alan W. Reichow., November/December, 2013.

Sensory Training Technology Takes Hold in the NFL

A Duke University study confirms stroboscopic technology offers improved vision for elite professional athletes and NFL players are turning to sensory performance training to improve their vision in an effort to up their game performance.

Stroboscopic Technology Enhances Visual Memory in Athletes

Sports often rely on the ability to keep fleeting information in memory (e.g., a basketball player making a no-look pass must remember the locations of his teammates and opponents), and any boost in visual memory abilities could manifest in improved performance. Previous research has shown that intermittent, or stroboscopic, visual training (i.e., practicing while only experiencing snapshots of vision) can enhance visual-motor control and visual cognition, yet many questions remain unanswered about the mechanisms that are altered. Another study used a partial-report memory paradigm to assess the possible changes in visual memory following training under stroboscopic conditions, exploring the impact of altering how visual information is accumulated over time by assessing how intermittent vision influences memory retention. In comparison to the control group, both stroboscopic groups (immediate and delayed retest) revealed enhanced retention of information in short-term memory, leading to better recall at longer stimulus-to-cue delays (640-2,560 ms). These results demonstrate that training under stroboscopic conditions has the capacity to enhance some aspects of visual memory, that these faculties generalize beyond the specific tasks that were trained, and that trained improvements can be maintained for at least a day.

### Memory SPEX

MEMORY SPEX are a non-invasive, drug-free treatment for memory loss attributed to Alzheimer's disease through stroboscopic technology. This technology simulates a strobe light effect but in an entirely different way from the conventional method. Unlike conventional strobe lights, which emit bursts of flashing lights (an invasive stimulus); MEMORY SPEX themselves do not produce any type of light whatsoever.

In fact, they produce their strobe-like effect by momentarily blocking out natural ambient light (a noninvasive stimulus) in much the same way as blinking does. By using LCD (Liquid Crystal Display) lens technology, MEMORY SPEX intermittently block ambient light by electronically switching the lenses from clear to opaque. No obtrusive light enters the eyes.

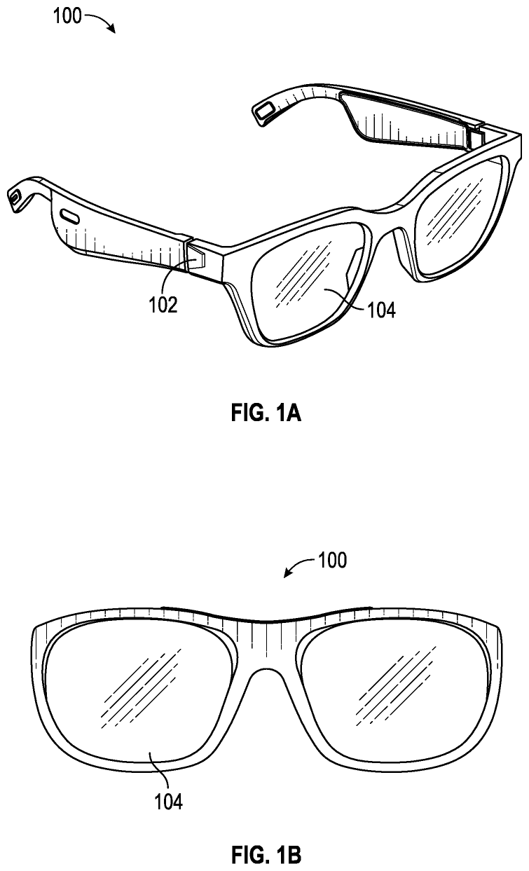

In embodiments, the MEMORY SPEX are incorporated into eyewear, as shown in FIGS. 1A and 1B, either eyeglasses or goggles that are easily put on and removed by the wearer. FIG. 1A shows an isometric view of an exemplary pair of stroboscopic eyewear 100. FIG. 1B shows a front elevation of the stroboscopic eyewear 100.

In embodiments, the LCD lenses 104 are flat and back-lit, having a colored LCD display.

Unlike conventional eyeglasses or goggles, the spectacles or goggles incorporating MEMORY SPEX are not passive optical devices that are simply worn. Because each “lens” is an LCD, they receive externally supplied power from, for example one or more batteries, such as hearing aid batteries or a built-in rechargeable lithium ion battery (provided in integrated electronic assembly 102). In embodiments, the MEMORY SPEX may be driven by other types of power source, solar energy, for example, or any other type of external power supply. In embodiments, the circuitry 102 that supports the LCD lenses may be located within the frame of the glasses or goggles.

Additionally, the functional capability of the MEMORY SPEX is mediated by one or more control programs that include computer-readable instructions for producing the previously described strobe-like effect. In embodiments, the control program may be hosted externally to the eyewear, for example on a mobile device communicatively coupled to the eyewear. In embodiments, the control program may be hosted and executed directly on the eyewear by means of a processing element such as a microcontroller provided in an integrated electronics assembly 102.

Various embodiments may include one of more of the following features, characteristics and options:
Lightweight and comfortable, only 2 oz. net weight;
Ruggedized to withstand ±40° C. temperatures and 7 g's of force
Hermetically sealed to resist moisture and sweat;
Manufactured to quality standards AS and ISO 9001;
Two-channel distraction control (visual and auditory);
Quickly and easily integrates with any existing training protocols;
Effective inside or outdoors, day or night, in all seasons and in any type of weather;
2-way data streaming via Wi-Fi;
Auditory streaming via Bluetooth for sound effects;
Multiple units controlled through secure iPad app or other tablet computer;
Individual units controlled through secure iPhone/Android app;
Programmable for individual needs or preferences;
Precise LCDs adjustable from 1 to 100 Hertz (Hz) (flashes per second) in 1 Hz increments;
Built-in rechargeable lithium-ion battery (integrated electronics assembly 102);

Completely wireless construction for maximum convenience;

Sleep button for instant pause or resumption of training (integrated electronics assembly 102); and
Expandable for video logging, heart-rate monitoring, telemetry and other distance functions.

### Visual Distraction

Visual distraction may be remotely controlled by a control program executing on an external mobile device, such as an iPhone, iPad or Android Phone communicatively coupled to the eyewear included in the integrated electronics assembly 102 over a wireless connection such as BLUETOOTH (IEEE 802.15.1, which is incorporated in its entirety as if fully set forth herein);
At least 10 levels of occlusion and single eye strobe capabilities;
As much as 20 percent greater opacity than has been provided by conventional devices;
Sweat resistant and anti-fog.

### Auditory Distraction

Auditory distraction may be remotely controlled from an external computing device over a wireless connection such as BLUETOOTH;
Holophonic sound effects that immerse the user in real life environments; and
Endless custom auditory applications (crowd noise, cheering/booing, etc.
   In embodiments, sound effects may be provided by one or more directional speakers, each having, for example, a frequency range of 20-20000 Hz, impedance 8.5Ω, Distortion less than 5.0% @ SPL (sound pressure level) 88 dB included in the integrated electronics assembly 102.

### Bluetooth Control Unit Features

Apple and android remote-control platform downloadable apps;
Standard pre-sets and unlimited custom programs;
Create a User Profile for keeping a progression log;
Data Retrieval: Ability to review past sessions and email directly to user.

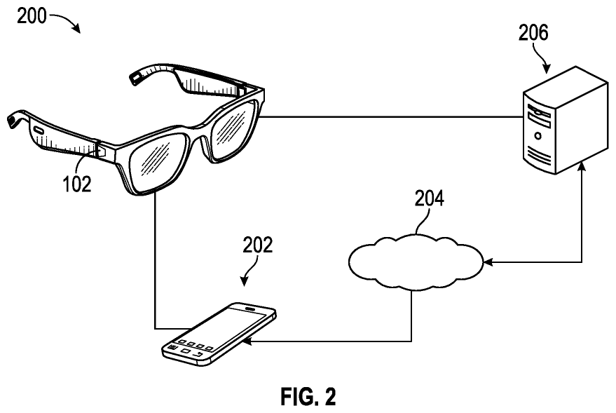

As previously described, the control device 202 and the eyewear may be communicatively coupled over a Bluetooth connection, as shown in FIG. 2. Accordingly, in embodiments, the eyewear 100 is provided with hardware for receiving and transmitting a radio signal according to the BLUETOOTH protocol in the integrated electronics assembly 102.

In embodiments, the eyewear 100 may transmit user data to the controller 202 via the BLUETOOTH connection, including frequency and length of usage, as well as frequency of strobe. In embodiments, the eyewear 100 may record and transmit data representing responsive brain activity to the control device 202.

In embodiments, the eyewear is equipped with a microcontroller within the integrated electronics assembly 102 for executing computer-readable instructions transmitted by either the control device or the remote server.

In embodiments, as shown in FIG. 2, the eyewear 100 is also communicatively coupled to a remote server 206 via a wireless connection 204 (for example, IEEE 802.11, the entirety of which is incorporated herein by this reference thereto). Patient data may be transmitted to the remote server 206 over the wireless connection 204 for access by healthcare professionals, so that care can be provided via remote access including treatment plans, care management and monitoring of progress as measured, for example, by neurological activity.

In a further embodiment, the eyewear 100 may constitute “smart glasses” having augmented reality (AR) capability.

In a further embodiment, the eyewear may use a patient's EEG (electroencephalogram) in combination with a brain-computer interface (BCI) as a biomarker for early disease detection and for tracking disease progression and for monitoring treatment progress. Early detection of Alzheimer's disease may be based on multimodal approaches; for example, augmenting longitudinal records based on nonlinear dynamics of the EEG and drug effects on the EEG dynamics. A BCI may constitute, for example OpenBCI components such as a Ganglion Board and a Headware Ultracortex Mark IV in conjunction with the eyewear and the audio device.

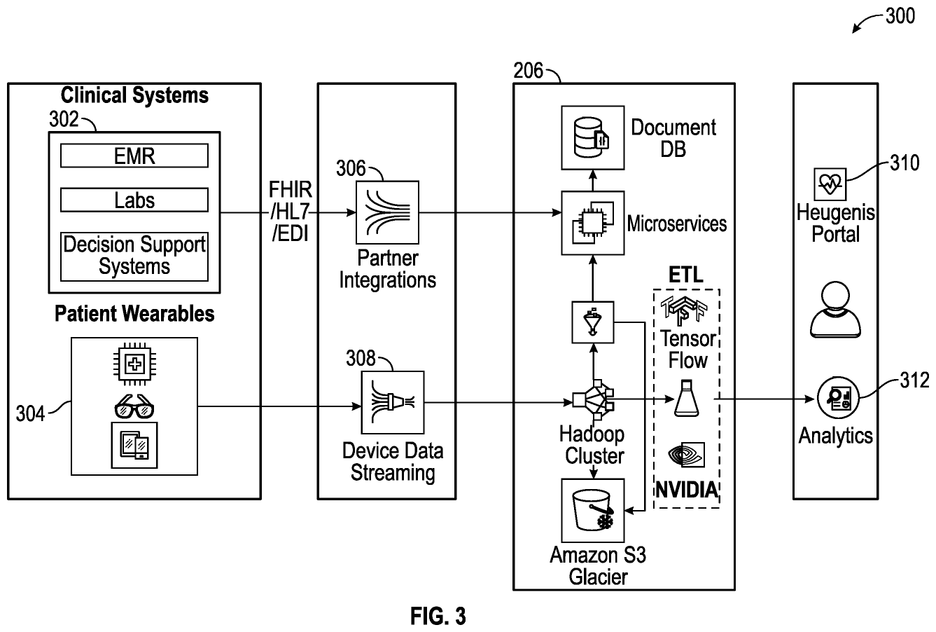

As shown in FIG. 3, the remote server 206 may host a doctor-patient portal 310, a component of the platform 300. In embodiments, the portal 322 provides at least one of the following features:
guided sessions to use the eyewear 100;
session insights to patients and caregivers;
personalized care plans;
metrics to track treatment progress; and
pharmacological and non-pharmacological treatment tracking.

In embodiments, the platform 300 provides one or more of the following provider services:
case management with workflows tailored to management of Alzheimer's disease (AD);
patient analytics 312;
a telemedicine connection with the patient;
a collaboration suite with the care team 306; and
integration of electronic medical records (EMR) 302.

In embodiments, the patient may access the portal 310 through a user interface hosted on the control device 202. In embodiments, the portal 310 may provide information, health tips, access to support groups and other services that enhance the quality of care.

Additionally, the doctor-patient portal 310 provides information and support to family members and caregivers. In embodiments, a caregiver portal provides tools such as protocols for basic care, providing direction and assurance, hope and peace of mind.

In embodiments, there is also a data link between the eyewear 100 and the remote server 206. In other embodiments, interaction of the eyewear 100 with the remote server 206 is mediated by the control device 202. A data link between the eyewear and the remote server enables healthcare providers to execute individualized treatment plans delivered to specific patient devices.

The platform 300 may be based on an open source API (application programming interface) that allows disparate wearable devices 304 to connect for each patient profile, allowing a consolidated data stream 308 to feed to a patient's personal health record (PHR) and external EMR 302 (electronic medical record) using the FHIR (fast healthcare interoperability resources) API. This also allows interface with healthcare applications for medical research and self-care based on platforms such as RESEARCHKIT and CAREKIT (APPLE, Inc., Cupertino Calif.).

The platform 300 allows care providers such as physicians to document and share prescriptive care plans: Using the platform 300, physicians can compose individualized care plans based on a proprietary decision support system that is incorporated within the platform 300. In embodiments, the individualized care plans may be stored in a document database housed on the remote server 206. Additionally, a plurality of microservices may also be served from the remote server 206. Automated intervention programs powered by an AI-based clinical decision support system support help social workers and caregivers to provide treatment with specific goals prescribed by the providers such as physicians. An ETL (extract, transform, load) module copies streamed device data to the document database and to an AI module for analytics.

Chronic care management: Exhaustive and personalized care management for patients is mediated through the portal 310.

The platform 300 provides care coordination by allowing collaboration between different organizations, creating care models that let accountable care organizations (ACOS) provide financial incentives to coordinate care between clinicians.

The fee-for-service system is the dominant payment mechanism for behavioral health services. The platform fosters 300 shared accountability across behavioral and medical systems to achieve good outcomes and to manage the broad care needs each individual requires.

Population analytics: The data generated by MemorySPEX and the SaaS platform 300 help to monitor effectiveness in providing quality care and taking corrective action. The physicians can use these data at an organizational level to analyze the various needs of a patient and to take action to increase available services. This also helps to provide better quality metrics showing patients are getting the care they need.

MemorySPEX can not only record patient's data to the portal 310 for doctor review, it can provide proof it is working through an accessory to the eyewear that can measure and report neurological activity in areas in the brain related to Alzheimer's disease, allowing doctors to remotely treat their patients.

In embodiments, the control device and the remote server 206 are also communicatively coupled via a wireless connection. In embodiments, patient data transmitted from the eyewear to the control device may be relayed to the remote server, providing still another avenue of access by health professionals to the patient data.

In embodiments, a software application downloaded to a mobile device 202 that is communicatively coupled to the eyewear 100 delivers a treatment protocol to the individual patient and measures, records and transmits all available data from the eyewear to the control device 202 (the mobile device), at least a portion of which is then transmitted to the remote server 206. In embodiments, the remote server 206 may transmit individualized treatment programs to the control device 202 instead of to the eyewear 100 as previously indicated.

In embodiments, the integrated electronics assembly 102 is provided with a power switch. Additionally, the integrated electronics assembly 102 may be provided with a port, such as a USB port for charging the power supply (USB 1.x, USB 2.0, with multiple updates and additions, USB 3.x, all of which are incorporated by reference as if fully set forth herein). In embodiments, a volume control may be provided to adjust the volume of sound emitted for auditory stimulation. In embodiments, a light that flashes to remind the patient that it is time for a treatment may be provided. The flashing light may be accompanied by an auditory signal.

In embodiments, hard-wired controls may be provided on the eyewear device 100 itself. In other embodiments, the device is software-controlled, with access to the individual controls being given through the UI of the control program residing on the control device 202.

It is to be appreciated that controls may be minimized in view of the fact that a large portion of the users of the stroboscopic device may be cognitively impaired to one degree or another.

This disclosure expands the use of existing technology to medical applications, providing the consumer with the choice to try a safe, non-drug and non-invasive therapy under the supervision of their doctor and in concert with existing drug regimens through use of the SaaS (software-as-a-service) platform 300 herein described.

The SaaS platform 300 provides a web application interface to create a new system of patient care that includes cost control and early intervention.

Data Aggregation from devices and multiple EHRs: The MemorySPEX portal 310 is based on an Open API interface that allows disparate wearable devices to connect for every patient profile, allowing consolidated data stream to feed to the personal health record (PHR) and external EMRs (Electronic Medical Records) using FHIR API. This also allows interface with https://www.apple.com/researchkit/ for clinical trials and sharing data with research institutions based on patient consent.

Document and Share Prescriptive Care plan: The SaaS platform 300 allows physicians to compose individualized care plans based on a proprietary decision support system that is part of the portal.

Automated intervention programs and goal oriented: Intervention programs powered by the clinical decision support system (AI) can help aid social workers and caregivers to aid treatment with specific goals prescribed by the physicians.

Chronic care management: Exhaustive and personalized care management for patients.

Care coordination: The SaaS platform 300 allows collaboration between different providers and creates care models that lets accountable care organizations (ACOS) provide financial incentives to coordinate care between clinicians. The fee-for-service system is the dominant payment mechanism for behavioral health services. The platform 300 fosters shared accountability across behavioral and medical systems to achieve good outcomes and to manage the broad care needs each individual requires.

Population analytics: The data generated by MemorySPEX and the SaaS portal 300 help to monitor their effectiveness in providing quality care and taking corrective action. The physicians can use these data at an organizational level to analyze various needs of a patient and to take action to increase available service. This will also help provide better quality metrics showing patients are getting the care they need.

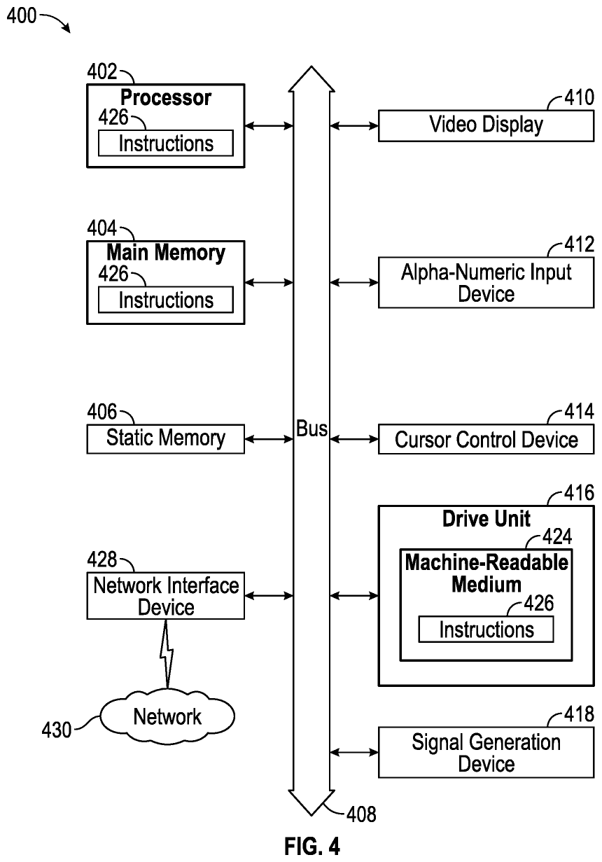

Referring now to FIG. 4, shown is a diagrammatic representation of a machine in the exemplary form of a computer system 400 within which a set of instructions for causing the machine to perform any one of the methodologies discussed herein below may be executed. In alternative embodiments, the machine may comprise a network router, a network switch, a network bridge, personal digital assistant (PDA), a cellular telephone, a web appliance or any machine capable of executing a sequence of instructions that specify actions to be taken by that machine.

The computer system 400 includes a processor 402, a main memory 404 and a static memory 406, which communicate with each other via a bus 408. The computer system 400 may further include a display unit 410, for example, a liquid crystal display (LCD) or a cathode ray tube (CRT). The computer system 400 also includes an alphanumeric input device 412, for example, a keyboard; a cursor control device 414, for example, a mouse; a disk drive unit 416, a signal generation device 418, for example, a speaker, and a network interface device 428.

The disk drive unit 416 includes a machine-readable medium 424 on which is stored a set of executable instructions, i.e. software, 426 embodying any one, or all, of the methodologies described herein. The software 426 is also shown to reside, completely or at least partially, within the main memory 404 and/or within the processor 402. The software 426 may further be transmitted or received over a network 430 by means of a network interface device 428.

In contrast to the system 400 discussed above, an embodiment uses logic circuitry instead of computer-executed instructions to implement the functionality of a device and method for stroboscopic light therapy for treatment of memory loss related to Alzheimer's disease.

Depending upon the particular requirements of the application in the areas of speed, expense, tooling costs, and the like, this logic may be implemented by constructing an application-specific integrated circuit (ASIC) having thousands of tiny integrated transistors. Such an ASIC may be implemented with CMOS (complementary metal oxide semiconductor), TTL (transistor-transistor logic), VLSI (very large-scale integration), or another suitable construction. Other alternatives include a digital signal processing chip (DSP), discrete circuitry (such as resistors, capacitors, diodes, inductors, and transistors), field programmable gate array (FPGA), programmable logic array (PLA), programmable logic device (PLD), and the like.

It is to be understood that embodiments of this system may be used as or to support software programs executed upon some form of processing core (such as the Central Processing Unit of a computer) or otherwise implemented or realized upon or within a machine or a computer-readable medium. A machine-readable medium includes any mechanism for storing or transmitting information in a form readable by a machine, e.g. a computer. For example, a machine-readable medium includes read-only memory (ROM); random access memory (RAM); magnetic disk storage media; optical storage media; flash memory devices; or any other type of media suitable for storing or transmitting information. Additionally, a “machine-readable medium” may be understood to mean a non-transitory medium. A non-transitory medium does not include a transitory, propagating signal.

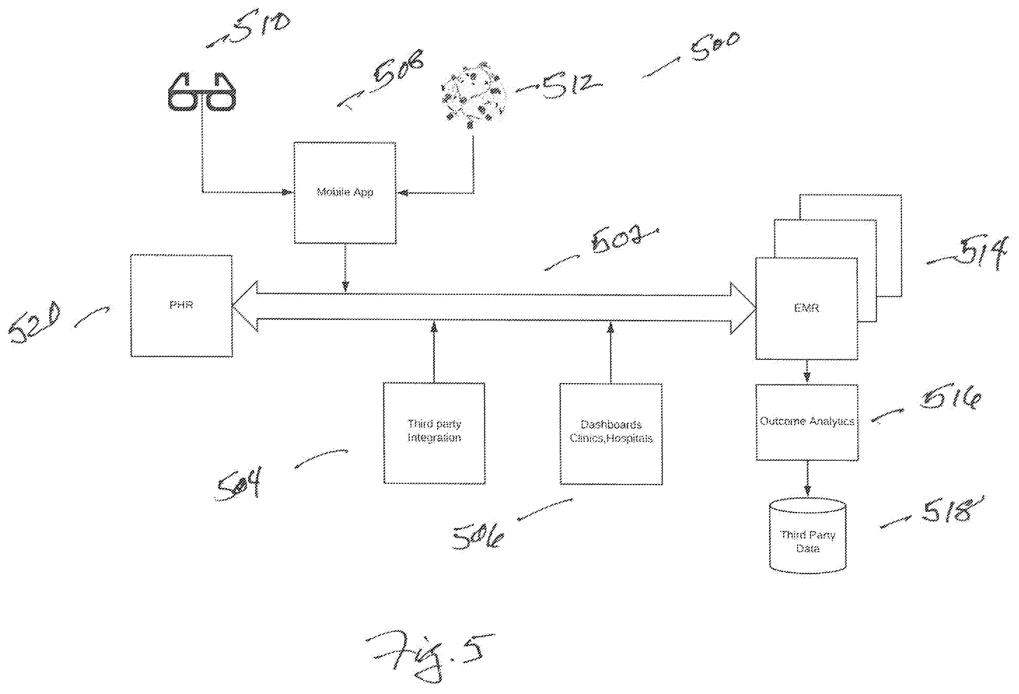

Referring now to FIG. 5, shown is a block diagram of a healthcare and Alzheimer's treatment management system 500 that may facilitate data collection, outcome analysis, decision support, third party data 518 and services integration 504, real time data collection and transfer, user interfaces, documentation and the like related to an AD (Alzheimer's disease) patient-related events from an ecosystem of devices under the control of a control application 508, including, but not limited to eyewear 510 and a headset-based 512 EEG monitoring. Various facilities 506 may be included in the system, for example caregiver facilities, clinics, hospitals, labs and imaging facilities, and other third-party facilities. Each facility may be connected together through a network 502 such as the Internet, a virtual private network, or the like, to facilitate real-time data capture and communication of data among the facilities.

The clinic facility may allow a doctor, physician, nurse or a caregiver to use the device on patients and enter any encounter data using, for example, an electronic computerized physician order entry system (CPOE). The facility may include an integration facility for automatically integrating data that may be captured in context of collecting patient device usage information and EEG in addition to the demographic and other related clinical data like health condition data, physical activity data, patient treatment data, patient oral consumption data, patient visitor data, patient outcome data, patient psychological data, patient dietary data, vital data for establishing a patient health record 520 in association with the facility EHR/EMR 514.

The outcome data may be utilized to determine the condition of the patient's health. An outcome analytics facility 516 may include a workbench that may implement one or more statistical techniques including, but not limited to, Multivariate analysis of variance (MANOVA), Multivariate analysis of covariance (MANCOVA), Discriminant analysis, Factor analysis, Canonical analysis, Mann-Whitney U-test, Kruskal-Wallis test, Wilcoxon signed ranks test, Friedman test, Spearman's rank order correlation.

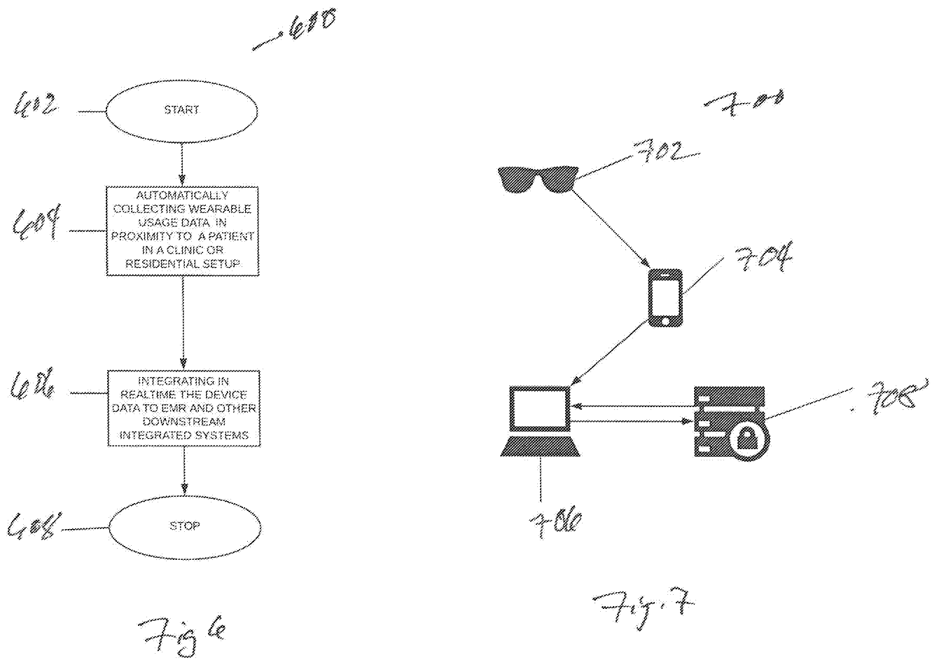

FIGS. 6 and 7 illustrate a process 600 and system 700 for automatically maintaining an EHR with device data for a patient that is collected during a medically-related event automatically and in real-time while in proximity to the patient. The process begins at step 602. At step 604, device data 702 related to the medically-related event associated with the patient may be collected. The step 604 of collecting the device data 702 of the medically-related event may be accomplished by an automatic data collection facility in proximity to the patient, such as those described herein. In embodiments, the device data 702 may be collected upon observation of an occurrence of a medically-relevant event. Further, the device data 702 may be identifiable by the device data in patient's EHR. Further, the automatic data collection facility may be a Bluetooth enabled Smart phone 704.

Further, the device data 702 may be raw device data that may be transformed to be suitable or used with a medication information database. As described herein, additional data about the medically-related event (which in embodiments may be the device data 702) may be fed into a computer 706. As an example, a caregiver may use the wearable to initiate the process 600. The coded information may be automatically read by the automatic data collection facility. The clinical facility 506 may also prompt the user to enter other relevant data about the patient's current condition at the time of medication administration. After the device data 702 of the medically-related event associated with the patient has been collected at step 604, the smartphone application 704 may communicate via an electronic communication, the device data 702, to the computer 706 which may represent any of the facilities depicted in FIG. 5 (e.g. clinical facility 106). In embodiments, the electronic communication may be the Internet. Thereafter, at step 606 the device data 702 and other information may be integrated automatically in real-time into the patient's EHR facility 708. The step 606 of integrating the device data 702 and other information may be accomplished by using the computer 706. The step 606 may further include using the computer 706 to transform the raw device data so that it can be integrated with the patient's EHR facility 708. The process ends at step 608.

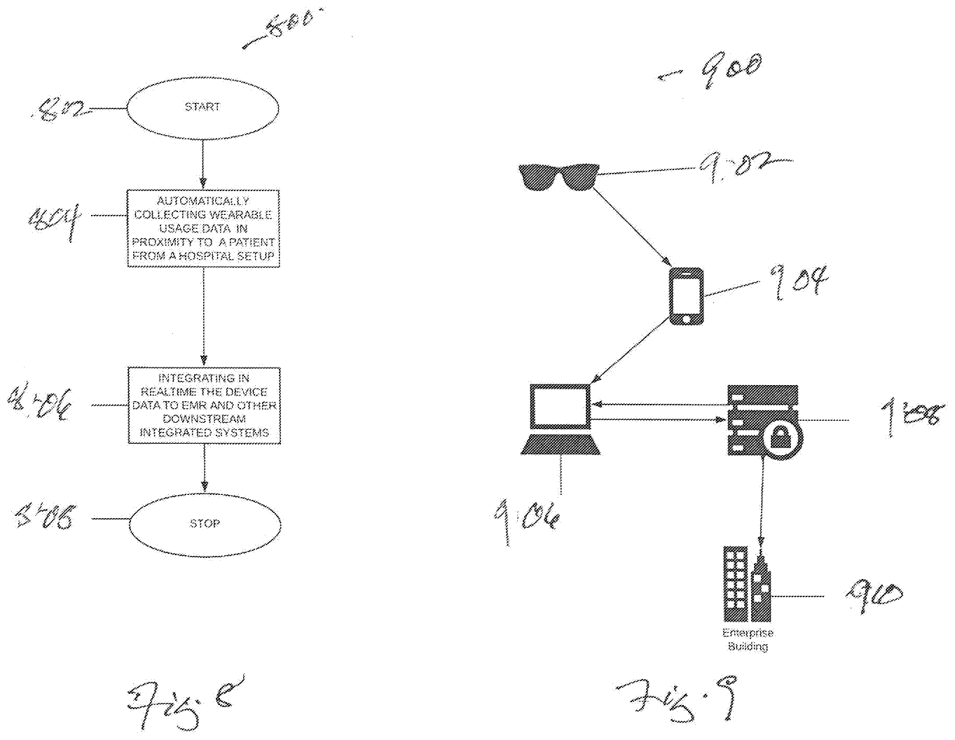

FIGS. 8 and 9 illustrate a process 800 and system 900 for automatically maintaining an EHR with device data for a patient that is collected during a medically-related event automatically and in real-time while in proximity to the patient in a hospital setup. The process begins at step 802. At step 804, device data 902 related to the medically-related event associated with the patient may be collected. The step 804 of collecting the device data 220 of the medically-related event may be accomplished by an automatic data collection facility 904 in proximity to the patient, such as those described herein. In embodiments, the device data 902 may be collected upon observation of an occurrence of the medically-relevant event. Further, the device data 902 may be identifiable by the device data in patient's HER 908. Further, the automatic data collection facility 904 may be, for example, a Bluetooth-enabled smart phone 904. Further, the device data 902 may be raw device data that may be transformed to be suitable or used with a medication information database. As described herein, additional data about the medically related event (which in embodiments may be the device data 902) may be fed into a computer 906. As an example, a caregiver may use the wearable 510 to initiate the process 800. The coded information may be automatically read by the automatic data collection facility 904. A clinical facility may also prompt the user to enter other relevant data about the patient's current condition at the time of medication administration. After the device data 902 of the medically-related event associated with the patient has been collected at step 804, the smartphone application 904 may communicate via an electronic communication, the device data 902, to the computer 906 which may represent any of the facilities depicted in FIG. 5. In embodiments, the electronic communication may be the Internet. Thereafter, at step 806 the device data 220 and other information may be integrated automatically with real-time into the patient's EHR facility 908 and a participating HIE (Health information exchange) simultaneously 910. The step 806 of integrating the device data 902 and other information may be accomplished by using the computer 906. The step 806 may further include using the computer 906 to transform the device data so that it can be integrated with the patient's EHR hospital facility 908 and other participating entities in a consortium that could be part of a HIE 910. The EHR users in other participating entities get comprehensive and relevant clinical information on the patient from device data which are not in their EMR through the HIE gateway. This allows the following:
Device (Wearable) data exchange from the EHR the HIE and vice versa;
Integrate the HIE data from other clinics within the EHR application and user workflow; and
Enrich patient data from related HIE solutions and services.
   The process ends at step 808.

In the foregoing specification, the invention has been described with reference to specific exemplary embodiments thereof. It will, however, be evident that various modifications and changes may be made thereto without departing from the broader spirit and scope of the invention as set forth in the appended claims. The specification and drawings are, accordingly, to be regarded in an illustrative sense rather than a restrictive sense.

1. A method of treating cognitive impairment resulting from neurodegenerative disease comprising:
providing a cognitively impaired subject with stroboscopic eyewear to wear during a treatment session, the stroboscopic eyewear including at least one flat LCD (liquid crystal display) lens;
supplying a first control signal to the eyewear that causes the at least one flat LCD lens to momentarily block ambient light;
exposing the subject to visual distraction by electronically switching the at least one flat LCD lens between states of clear and opaque according to the control signal at a predetermined frequency; and
exposing the subject to a predetermined number of treatment sessions in a day, each lasting for a predetermined time interval.

2. The method of claim 1, wherein the predetermined frequency occupies a range of 20-60 Hz.

3. The method of claim 1, wherein the predetermined time interval is twenty minutes.

4. The method of claim 1, wherein the predetermined number of treatment sessions in a day is at least one.

5. The method of claim 1, further comprising:
supplying a second control signal to the eyewear that exposes the subject to auditory distraction via an auditory channel included in the stroboscopic eyewear that streams programmed sound effects

6. The method of claim 5, wherein the first and second control signals are transmitted to the eyewear from a Bluetooth transmitter and received by a Bluetooth receiver incorporated into the eyewear.

7. The method of claim 1, wherein the LCDs are adjustable across a range of 1-100 Hz.

8. The method of claim 1, wherein said eyewear provides:
ten levels of occlusion; and
single eye strobe capabilities/

9. The method of claim 1, further comprising programming said wherein the eyewear to individual needs and desires.

10. The method of claim 1, further comprising:
transmitting user data to a remote server for evaluation by a healthcare professional.

11. A platform for treating cognitive impairment resulting from neurodegenerative disease comprising:
stroboscopic eyewear for exposing a subject to sensory distraction during a treatment session, the stroboscopic eyewear comprising:
an eyewear frame;
at least one flat LCD lens fixedly disposed within said eyewear frame;
a wireless receiver fixedly disposed within said eyewear frame;
a wireless network adaptor fixedly disposed within said eyewear frame;
a control device wirelessly coupled to the eyewear transmitting computer-readable control instructions to the eyewear for:
causing the at least one flat LCD lens to momentarily block ambient light;
exposing the subject to visual distraction by electronically switching the at least one flat LCD lens between states of clear and opaque according to the control signal at a predetermined frequency; and
exposing the subject to a predetermined number of treatment sessions in a day, each lasting for a predetermined time interval;
an API (application programming interface) that allows disparate wearable devices to connect for each patient profile, allowing a consolidated data stream to feed to a patient's personal health record (PHR) and external EMR,
allowing interface with healthcare applications for medical research and self-care;
and
a remote server receiving patient data transmitted from said eyewear.

12. The platform of claim 11, further comprising separate channels for vision and auditory distraction.

13. The platform of claim 11, wherein said wireless connection enables 2-way data streaming between said eyewear and either of the mobile device and the remote server.

14. The platform of claim 11, wherein said LCD lenses are adjustable between 1-100 Hz at 1 Hz intervals.

15. The platform of claim 11, further comprising a sleep button for instant pause or resumption of training.

16. The platform of claim 11, wherein the instructions include instruction for providing multiple levels of occlusion and single eye strobe capabilities

17. The platform of claim 11, wherein the instructions include instructions for providing any of:
single eye strobe capabilities; and
alternating eye strobe capabilities.

18. The platform of claim 11, wherein said treatment data is streamed from the eyewear device to a doctor-patient portal hosted, a least in part on said remote server.

19. The platform of claim 18, further comprising an artificial intelligence module for processing patient data streamed from said eyewear device to said doctor-patient portal.

20. The platform of claim 18, wherein said doctor-patient portal includes at least one of the following features:
an instruction module for use of the eyewear;
a module for capturing treatment session data;
a module for creating and disseminating personalized care plans;
a module for calculating metrics to track treatment progress;
a module for pharmacological and non-pharmacological treatment tracking;
a case management module;
a patient analytics module;
a telemedicine connection with the patient;
a collaboration module for collaboration with members of a care team; and
integration of electronic medical records (EMR).
